(19) United States
(12) Patent Application Publication
(10) Pub. No.: US 2011/0004176 A1
(43) Pub. Date: Jan. 6, 2011

(54) MATERIAL WEB FOR USE IN AN ABSORBENT ARTICLE

(75) Inventors: Patrik Andersson, Billdal (SE); Anders Persson, Hammaro (SE)

Correspondence Address: BUCHANAN, INGERSOLL & ROONEY PC, POST OFFICE BOX 1404, ALEXANDRIA, VA, 22313-1404, US

(73) Assignee: SCA Hygiene Products AB, Goteborg (SE)

(21) Appl. No.: 12/865,743

(22) PCT Filed: Feb. 19, 2008
(86) PCT No.: PCT/SE08/50193

§ 371 Date: Aug. 2, 2010

(51) Int. Cl. A61F 13/45; B32B 7/02; B32B 3/24; B32B 3/30; B29C 65/08; B32B 38/04

(52) U.S. Cl. 604/378; 428/132; 156/73.1

(57) ABSTRACT

A material web includes at least one fibrous material layer and has a first and a second surface. The material web comprises recesses in the first surface, which recesses have a diminishing cross-sectional area along at least a part of their extension in a direction towards the second surface. The material web further comprises recesses in the second surface, which recesses have a diminishing cross-sectional area along at least a part of their extension in a direction towards the first surface, and which form pairs with opposite recesses in the first surface. The recesses in at least some of the pairs are connected to each other via at least one hole. A method for producing such a material web and an article comprising such a material web is also provided.

### TECHNICAL FIELD

The present invention concerns a material web for use in an absorbent article, which material web comprises at least one fibrous material layer and has a longitudinal direction, a transverse direction and a thickness direction as well as a first and a second surface, which surfaces are situated on opposite sides of the material web and of which one surface is intended to face towards the user of the article while the opposite surface is intended to face away from the user of the article. The material web comprises recesses in the first surface with an extension in the thickness direction of the material web, which recesses have a diminishing cross-sectional area along at least a part of their extension in a direction towards the second surface. The present invention also concerns an absorbent article comprising such a material web and a method for producing such a material web.

### BACKGROUND ART

Absorbent articles which are intended for single use usually comprise a liquid-permeable surface layer, which faces towards the body of the user during use. A surface layer of this type is often constituted by a nonwoven material, i.e. a fibre material in which the constituent fibres have been bonded together in some way other than weaving, knitting or similar methods which give a regular fibre arrangement.

It is also known to arrange a liquid transfer layer between the surface layer and an absorbent body contained in the article. A liquid transfer layer of this type should have the ability to quickly receive large quantities of liquid and spread the liquid, and also temporarily store the liquid before it is absorbed by the absorbent body below. This is of great importance, especially in the case of modern slim compressed absorbent bodies, which often have a high content of superabsorbents. Such materials have, certainly, a high absorption capacity; however, in many cases they have an acquisition rate that is much too low to be able to instantaneously absorb the large quantity of liquid which can be emitted in only a few seconds during urination. A porous, relatively thick liquid transfer layer, for example in the form of a fibrous wadding, a bonded or unbonded carded fibre layer or some other form of fibre material, has high instantaneous liquid acquisition capacity and can temporarily store the liquid until it can be absorbed by the absorbent body. These circumstances also apply to porous foam material. For the absorbent article to be able to receive repeated volumes of liquid, the liquid transfer layer must have time to be essentially emptied of liquid between each wetting. The porous structure of the liquid transfer layer thus suitably works in combination with a denser and/or more hydrophilic absorbent body.

The liquid transfer layer and the liquid-permeable surface layer can be joined together when heated to form a material web in the form of a laminate by using, for example ultrasound or hot calendering. At least one of said liquid transfer and surface layers comprises a thermoplastic material, which melts on heating and bonds together the two layers. At the bonds, recesses are formed in the laminate, giving the laminate a three-dimensional surface structure. However, the material webs obtain a more or less liquid-impermeable character at the bottoms of the recesses, which would lead to reduced liquid-permeability at the bonds. Thus, liquid received by the article will gather in the recesses and not be led on into the underlying absorbent structure.

A solution to the above-mentioned problem is to replace the recesses with penetrating holes which extend all the way through the laminate. Such holes can be produced by, for example, passing the two layers in the laminate between two binding rolls, one of which is provided with spikes, which penetrate the layers while they are being heated and bonded together in order to produce a bonded laminate with penetrating holes. However, perforating by means of spikes requires that the material layers are fed forward at low speed, especially if stable, round holes are desired, and the slow processes result in expensive materials. This is a particularly significant problem where the manufacture of disposable articles is concerned, as the cost of materials is very important. The complexity of the manufacturing process and the wear and tear it puts on the component parts of the manufacturing device also contribute to increasing the production costs.

Penetrating holes can also be produced without spikes or similar tools, for example by means of ultrasound. A faster process, and consequently a reduction in material costs, are thus achieved. However, the material web created in this way displays a less stable three-dimensional structure, a relatively low tensile strength and poorer bonding of the layers in the material web.

Another solution to the problem is to create recesses with small holes formed in them, which holes lead the liquid down into the material web. This is known from, for example WO 93/11725, in which a heated head having a bonding surface provided with needles bonds an upper and a lower sheet to form a laminate, thus forming apertured recesses at the bonds. However, these holes only extend through the upper sheet and a certain distance down into the lower sheet.

Recesses provided with holes can, of course, also be formed in material webs comprising only one layer. A material web of this kind is described in WO 2007/035038 A1, in which a liquid transfer layer is provided with recesses, the bottoms of which recesses are in turn each provided with a penetrating hole. The hole and the recess are formed by means of a heated needle. However, these solutions also lead to high production costs due to low production speeds, complex manufacturing processes and high wear and tear on components.

Moreover, the holes in the above-described embodiments indeed give the material web greater liquid-permeability but at the same time they allow insufficient distribution of the received liquid over underlying layers in the article. In addition, there is a risk of rewetting of the surface of the material web that faces towards a user of the article, as liquid present further inside the article forces its way up through the holes.

A first object of the present invention is to obtain a material web which displays good liquid-permeability and a stable, three-dimensional structure while at the same time giving good distribution of liquid over underlying layers and counteracting rewetting of the material web.

A second object of the present invention is to obtain an article comprising such a material layer.

A third object of the present invention is to achieve a method for creating such a material web at a low production cost.

### SUMMARY OF THE INVENTION

The first object is achieved by means of a material web for use in an absorbent article. The material web comprises at least one fibrous material layer and has a longitudinal direction, a transverse direction and a thickness direction as well as a first and a second surface, which surfaces are situated on opposite sides of the material web and of which one surface is intended to face towards a user of the article while the opposite surface is intended to face away from a user of the article. The material web further comprises recesses in the first surface with an extension in the thickness direction of the material web, which recesses have a diminishing cross-sectional area along at least a part of their extension in a direction towards the second surface. The material web also comprises recesses in the second surface with an extension in the thickness direction of the material web, which recesses have a diminishing cross-sectional area along at least a part of their extension in a direction towards the first surface, and which recesses form pairs with opposite recesses in the first surface. The recesses in at least some of the pairs are connected to each other via at least one hole.

The holes and the recesses thus form hour-glass shaped hollows or channels through the material web, which give the material web high liquid-permeability. This liquid-permeability is further increased by the diminishing form of the recesses in the first surface, which results in capillary forces leading received liquid in a direction towards and down into the holes. The presence and shape of the recesses in the second surface contribute in turn to distributing the received liquid over underlying layers in the article, while the cavity created by the recesses prevents liquid present in the absorbent core of the article from rewetting the first surface via the holes. In addition, the recesses and the holes contribute to good ventilation of the material web and the article.

The recesses also contribute to giving the material web a stable, three-dimensional structure and high tensile strength. This is due to the recesses and holes being formed in a two-step process, which is described in more detail later and which comprises the first step of forming recesses in the material web, whereupon the material web acquires a more or less film-like structure at the bottoms of the recesses. In the second step, the holes are then formed in the film-like structure at the bottoms of the existing recesses, whereupon the film-like structure partly remains and gives the finished material web the desired stable, three-dimensional structure and material strength.

Note that the recesses can have a diminishing cross-section over all or only parts of their extension and that they can thus have, for example, a cylindrical form over parts of their extension.

With regard to the extension of the recesses in the thickness direction of the material web, this can, of course, vary from case to case. In general, the recess on one side of the material web has a greater extension in the thickness direction of the material web than the recess on the opposite side of the material web, which means that the waist of the hour-glass will usually be located closer to one of the sides. For example, recesses formed by means of ultrasound will usually be deeper on the side that faces away from the ultrasonic horn. It can be suitable to form the material web in such a way that the larger recesses are located on the side that faces towards the user during use of the article comprising the material web, and that will thus receive faeces and body fluids, which it can be desirable to store temporarily in the recesses. However, it is possible to let the waist of the hour-glass be in the middle of the material web or closer to the surface that is intended to face away from the user.

As regards the more or less film-like structure at the bottoms of the recesses, the distance it extends into the material web can vary. The extent of the film-like structures in the thickness direction of the material web depends, for example, on which materials are included in the material web, how much energy is applied to the material web in the bonding step and how much time the bonding step takes. The film-like structures at the bottoms of a pair of mutually opposite recesses may thus be present only at the bottoms of the recesses, just as they may be joined and form a continuous film-like structure.

As mentioned above, it is desirable that the material web has good liquid-permeability. This is especially the case when the material web is intended to be used in a product that is intended to receive large quantities of urine in a short time, such as an incontinence protector. Such good liquid-impermeability is suitably achieved by making the total cross-sectional area for the hole or holes that connect a recess with an opposite surface of the material web sufficiently large. A large total cross-sectional area is achieved by either a single hole with a large cross-sectional area or several holes with a smaller cross-sectional area connecting said recesses. The latter alternative is particularly advantageous as, in addition to achieving good liquid-permeability for the material web, it is desirable to let the recesses in the first surface function as spaces for temporary storage of received faeces and more viscous body fluids. The holes are then formed with such a cross-sectional area that the remaining material in the bottom of the recesses forms a net which only allows low-viscous body fluids, such as urine, to penetrate to an underlying absorbent body. The same effect can of course be achieved with only one hole formed in the bottom of the recess but as a result of a lower liquid-permeability in the recess. Similarly, it can be desirable to prevent pulp, fibres and particles from an absorbent core in an article comprising said material web from being forced out from the article through the penetrating holes in the material web. This, too, can suitably be prevented by forming each of the holes through the material web with such a cross-sectional area that they do not allow said pulp, particles and fibres to pass through. Further advantages of forming several small holes in the apertured recesses are that small holes give a visually attractive product, which gives the impression of being able to retain applied liquid, and also that the formed net structure gives a particularly good tensile strength to the material web.

If the material web comprises at least a first and a second material layer, of which at least one comprises a thermoplastic material, it is advantageous if the thermoplastic material during the forming of the recesses is made to at least partly soften and thus bond together the two material layers at the recesses. The number of production steps is thus reduced, giving lower production costs. As is described below, the two-step method ensures that the layers are well bonded together despite the material web being perforated, as a part of the bonded structure which is formed in the first step remains after perforation in the second step.

It is possible to form holes within a first zone of the material web with an average cross-sectional area that is smaller than the average cross-sectional area for holes within a second zone of the material web. By this means, a material web can be obtained, which, for example, is adapted within the first zone to receive low-viscous body fluids and within the second zone is adapted to receive faeces. Obviously, it is suitable if the recesses also have corresponding differences in size, so that the recesses within the first zone are adapted for temporary retention of body fluids while the recesses in the second zone are adapted for temporary retention of faeces. It should be understood that a material web can have more than one zone and also that the average density between the recesses can vary from zone to zone.

It is of course possible to provide only some of the recesses with holes, just as it is possible to provide a majority of, or all of the recesses with holes. Recesses without holes can, for example, be situated outwith the area that is intended to be liquid-permeable, or have a mainly decorative function. Usually, the bonds which lie at the side edges of the material web are those that should not be liquid-permeable and it is therefore particularly advantageous if the recesses provided with holes are situated within a part that is situated centrally in the transverse direction of the material web. An example of a way to achieve the forming of holes in only some of the recesses is for the device that creates the holes, for example a patterned roll or an ultrasonic horn, to have a different extension in the transverse direction of the material web than the device that creates the recesses.

The second object is achieved by means of an absorbent article in accordance with claim 7, comprising a material web of the type described above. The person skilled in the art would understand that the material web can be arranged in a number of different places in the article and the said first surface can face towards either the side of the article that is intended to face a user during use, or the side of the article that is intended to face away from the user. According to one preferred embodiment, the material web constitutes a liquid-permeable surface layer in the article, wherein the first surface suitably faces outwards in the article and constitutes a surface which is intended to face towards the user during use. The material web can also comprise a liquid transfer layer situated under the surface layer. It is also advantageous if the material web comprises zones of the type described above, wherein the material web is suitably orientated in the article in such a way that the zone intended to receive low-viscous body fluids is closer to a front edge on the absorbent article, while the zone that is intended to receive faeces is closer to a rear edge on the article. As has been mentioned above, the apertured bottoms in the material web have two main functions, i.e. to let liquid into the article and to prevent fibres and particles from falling out of the article. Therefore, it is advantageous if each recess contains two or more smaller holes instead of one larger hole. The bottom of the recess thus acts as a filter which allows liquid to pass through, but not particles and other solid or highly viscous substances. This means that the material web also acts as a means of separating faeces from other body fluids. At the same time, the holes ensure that the material web obtains good liquid-permeability, which is particularly desirable when the material web is used in a product that is intended to receive large quantities of urine in a short time, such as an incontinence protector.

The third object is achieved by means of a method in accordance with claim 10 for producing an apertured structure in a material web for use in an absorbent article. The material web comprises at least one fibrous material layer and has a longitudinal direction, a transverse direction and a thickness direction as well as a first and a second surface, which surfaces are situated on opposite sides of the material web and of which one surface is intended to face towards a user of the article while the opposite surface is intended to face away from a user of the article. The method comprises the first step of forming pairs of mutually opposite recesses in the first and second surfaces, which recesses have an extension in the thickness direction of the material web, the recesses in the first surface being formed with a diminishing cross-sectional area at least along part of their extension in a direction towards the second surface and the recesses in the second surface being formed with a diminishing cross-sectional area at least along part of their extension in a direction towards the first surface, and the second step of forming holes in the material web, which holes each connect two recesses belonging to a pair.

Forming the recesses and the holes in separate steps enables the supplying of energy to be optimised at each step. Thus, it becomes possible during the forming of the recesses, which is done in the first step, to optimise the energy supply at the bottoms of the recesses in such a way that existing thermoplastic components in the fibre structure at the said bottoms melt or are softened and form bonds between the fibres. Thus, the material web at the bottoms of the recesses will have a more or less film-like structure, which gives the material web a stable, three-dimensional structure and good tensile strength. Similarly, it will be possible in the subsequent penetration step to optimise the energy supply in such a way that at least some of the said recesses are connected by means of one or several penetrating holes. Some of the bonded, film-like fibre structure will then remain even after the perforation step, so that the finished material web obtains the desired stable, three-dimensional structure and material strength. This is considerably different to previous binding and perforation methods in which the forming of the recesses and the holes has been carried out in one step. When the binding and the perforation are carried out at the same time, so much energy is used to make holes in the material that the material at the bonds is burnt away. This is a particular problem in ultrasound processes and results in poor binding of the layers in the material web and also the material web obtaining a low material strength and a less stable three-dimensional structure.

Consequently, the two-step method means that the material web is provided with penetrating holes in existing recesses, which gives the material web good liquid-permeability compared with material webs which have only recesses.

Furthermore, the two-step method described above can be carried out with low productions costs. This is achieved above all due to the fact that the two-step method, by permitting an optimisation of the energy supply in each step, enables the creation of a stable, apertured structure using techniques, such as ultrasound and hot calendering, which allow the material web to be fed forward at high speed. By this means, not only a high production speed but also a possible synchronisation of the different process steps are achieved, making it possible to manufacture the material webs in-line. The above-mentioned techniques also contribute to low production costs by causing less wear and tear on the manufacturing device. However, it should be pointed out that lower production costs do not necessarily require the use of ultrasound or hot calendering. The two-step method also gives lower production costs when other techniques are used, wherein the recesses and holes are formed by means of raised portions and needles penetrating the material web, as the two-step method permits an optimisation of the pressure that is applied in each step and thus reduced wear and tear on the manufacturing device.

Ultrasound and hot calendering methods also result in the advantages that the manufacturing method becomes less complex and more flexible, for example with regard to the choice of size and location of the holes and recesses, and also with regard to the choice of which recesses are to be provided with holes.

As is mentioned above, it is advantageous if both steps in the above-described two-step method are carried out by means of an ultrasonic welding device comprising an ultrasonic horn, as this technique gives particularly high production speed, great flexibility and less wear and tear on the manufacturing device. It is especially suitable if the first step is carried out using an ultrasonic horn with a smooth surface facing towards the material web and if the second stage is carried out using an ultrasonic horn with a patterned or knurled surface facing towards the material web, as a patterned or knurled surface is particularly advantageous in obtaining a good effect during perforation. However, a good result can also be achieved using hot calendering, which similarly gives high production speed, great flexibility and less wear and tear on the manufacturing device. Regardless of which technique is used, it is advantageous to use the same technique for creating the holes and the recesses, as this enables a reduction in the number of elements in the manufacturing device and, consequently, lower production costs. However, it should be understood that the scope of protection is not limited to ultrasound and hot calendering and that different techniques can be used for creating the holes and the recesses.

If it is the case that an ultrasonic horn is used to create both the recesses and the holes, it is advantageous if said ultrasonic horn works against one and the same roll. In this way, a further reduction of the number of elements in the manufacturing device is obtained, which leads to a reduction in production costs compared with using a separate roll for each horn, which is an alternative, but less preferred, possibility. This is also a means of avoiding the synchronisation problems which arise when the material web is to be put onto a second roll prior to forming the holes. In other words, it ensures in a simple manner that the penetrating holes are actually formed in the recesses, thus increasing the quality of the final product. In the same way, it is also advantageous in the case of hot calendering if the forming of the recesses and the holes is carried out against one and the same roll.

### BRIEF DESCRIPTION OF DRAWINGS

The invention will now be described in more detail with reference to the attached drawings, in which

FIG. 1 shows a perspective view of a material web according to the invention,

FIG. 2 shows a cross section along the line II-II through the material web in FIG. 1,

FIG. 3a shows a view from above of a bond according to the invention,

FIG. 3b shows a view from above of a bond according to an alternative embodiment of the invention,

FIG. 4 shows a device for manufacturing a material web according to the invention,

FIG. 5a shows an embodiment of the ultrasonic horn in FIG. 4,

FIG. 5b shows an alternative embodiment of the ultrasonic horn in FIG. 4, and

FIG. 6 shows a view from above of an absorbent article comprising a material web according to the invention.

### DESCRIPTION OF EMBODIMENTS

The term material web in the present invention denotes a web comprising one or several layers. For example, a material web can constitute, as described below, a surface layer and a liquid transfer layer in an absorbent article. In addition, a layer can comprise one or several strata.

The term film-like structure denotes a structure comprising melted or softened thermoplastic components. The film-like structure suitably comprises intact fibres, which are bonded together by the melted or softened thermoplastic components and give strength to the welded joints. However, it is also conceivable that all fibres in the film-like structure have been melted. The amount of intact fibres can vary greatly from case to case depending on, for example, which materials are included in the material web, how much energy is applied to the material web in the binding step and how much time the binding step takes.

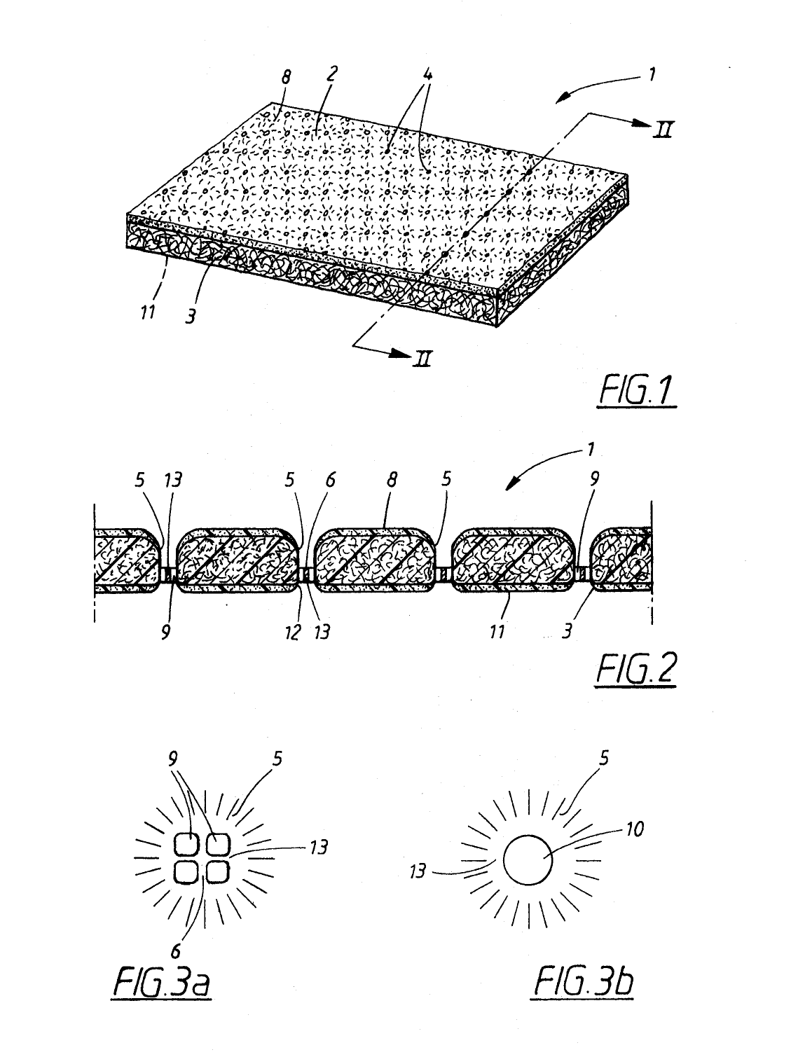

FIG. 1 shows a material web in the form of a laminate 1 comprising a first fibrous, liquid-permeable material layer 2, which serves as a surface layer and a second fibrous, liquid-permeable material layer 3, which serves as a liquid transfer layer. The laminate 1 has an extension in one plane and has a longitudinal direction and a transverse direction in said plane as well as a thickness direction perpendicular to the plane. The laminate 1 also has a first and a second surface 8, 11.

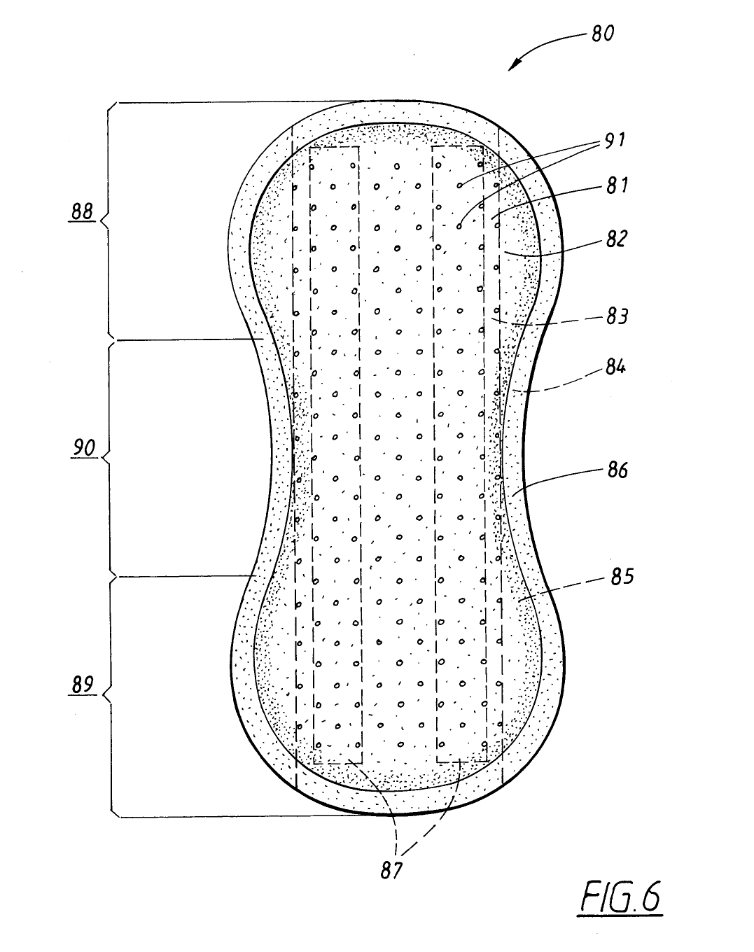

The laminate is intended for use as a liquid-permeable surface material in an absorbent article 80 (FIG. 6), where the surface layer 2 is intended to face towards a user of the article 80. The surface layer 2 should therefore have a soft, non-chafing surface facing towards the user and is advantageously produced from a relatively thin nonwoven material.

Nonwoven material can be produced using many different methods, for example by carding or spinning a fibre gauze, which is then bonded. Furthermore, so-called melt-blown technique can be used to deposit short fibres in the form of a fibre web. There are a number of different ways of bonding fibres in a nonwoven material. For example, different types of bonding agent can be used. In addition, heat-meltable components in the material can be used for bonding with ultrasound or by applying heat. Other bonding methods are needling and hydro-entangling. Different bonding methods can also be combined with one another. A particularly common nonwoven material is spunbond nonwoven.

The liquid transfer layer 3, which advantageously has a greater extension in the thickness direction of the laminate 1 than the surface layer 2, can in turn consist of one, two or several strata of different or similar types of material, for example a porous, resilient fibre material. The liquid transfer layer 3 should have the ability to receive large amounts of liquid in a short time, spread the liquid in the plane of the layer, transport the material to an absorbent body arranged under the laminate, and also be able to temporarily store liquid that has not had time to be absorbed by the absorbent body. The following materials are particularly suitable for use in the second layer: synthetic fibre wadding, carded, bonded or unbonded fibre layers, or bulky nonwoven materials. A special type of fibre material that can be used is known as tow, which is understood to mean mainly parallel, long or endless fibres or fibre filaments, which are arranged in the form of unbonded layers or strands. Another type of suitable material is porous hydrophilic foam materials.

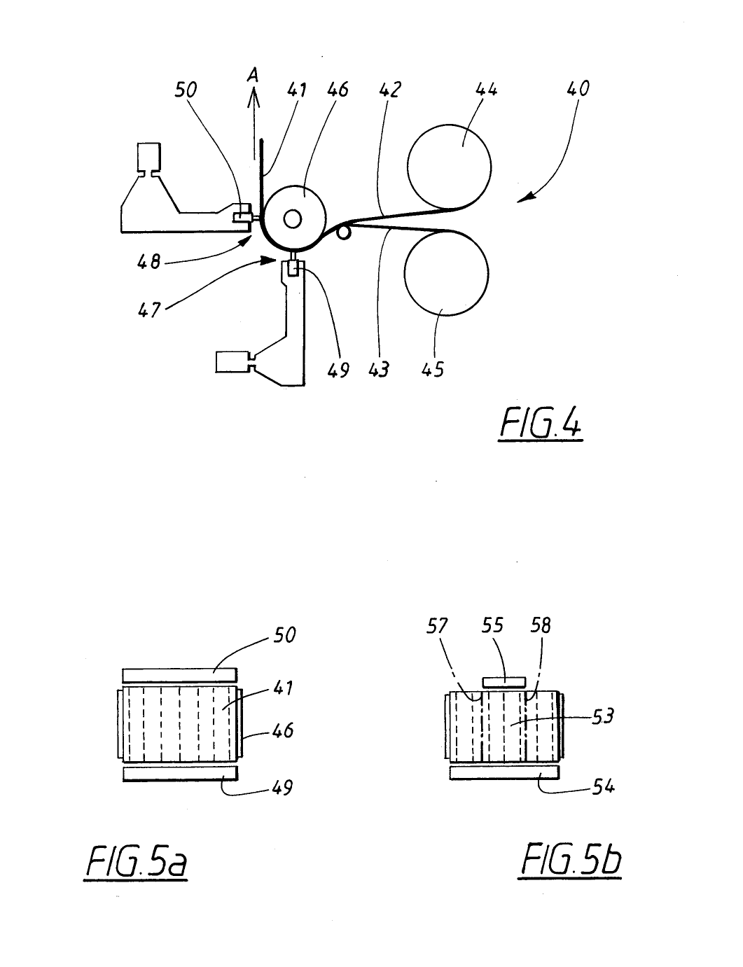

The two layers 2, 3 are mutually connected at a large number of bonds 4, which have been formed by means of a method which is described in more detail below with reference to FIG. 4. At least the surface layer 2, but preferably both layers 2, 3, comprise thermoplastic material. Suitable thermoplastic materials are polyolefines, such as polythethylene and polypropylene, and also polyamides, polyester, and the like. Different types of mono-, bi- and polycomponent fibres can also be used, as can various polymer mixtures. The bonds 4 have been formed by simultaneously compressing and applying energy to the laminate 1, whereupon the thermoplastic material has been caused to soften or melt at the bonds 4. When the thermoplastic material cools, it hardens and serves as a bonding agent for the layers 2, 3 in the laminate 1. Moreover, the compression of the porous structure in the layers 2, 3 creates pairs of mutually opposite recesses 5, 12 in a first and a second surface 8, 11 in the laminate, which recesses 5, 12 give the first and second surfaces 8, 11 a wave-like structure (FIG. 2). The recesses 5, 12 have an extension in the thickness direction 1 of the laminate and are separated by an intermediate wall 13. As can be seen in FIG. 2, the recesses 5 in the first surface 8 have a diminishing cross-sectional area in a direction towards the intermediate wall 13, which provides better liquid transfer properties into the article and also prevents rewetting of the surface layer 2. The recesses 12 in the second surface 11 also have a diminishing cross-sectional area in the direction towards the intermediate wall 13, which results in better distribution of liquid to underlying layers. The recesses 12 also counteract rewetting of the surface layer 2, as liquid in underlying layers must pass the cavity created by the recesses 12 in order to reach the surface layer 2. Furthermore, the recesses 12 are conducive to good ventilation of the article. The fusing together of the thermoplastic materials gives the intermediate wall 13 a film-like structure, which gives stability to the three-dimensional structure of the laminate 1. However, the film-like structure gives the intermediate wall 13 a more or less liquid-impermeable character, as the liquid-permeability is negatively affected by a higher proportion of melted or softened thermoplastic material. As it is desirable to obtain high liquid-permeability for the laminate, the recesses 5, 12 in each pair are therefore connected via several penetrating holes 9, which thus connect the first surface 8 with the second surface 11. These holes 9 are intended to transport liquid to the underlying layers in the absorbent article. Moreover, the concentrated fibre structure that has arisen around the bonds as a result of the compression which occurs during the joining process, results in the area immediately around each bond 4 having finer capillaries than the surrounding material, which further contributes to increasing the liquid transfer capability from the first to the second layer.

As can be seen from FIG. 1, the laminate 1 has point-like bonds, which form a bonding pattern. However, it should be understood that the bonds 4 and also the recesses 5, 12 at the bonds 4 can have any form. For example, they can have a line-shaped, circular or oval cross-section in the plane of the laminate. In the same way, the penetrating holes 9 can have another form than those shown in FIGS. 3a and b; for example, they can have a circular, oval, line-shaped or square cross-section in the plane of the laminate. Moreover, the bonds 4 in FIG. 1 are relatively homogeneously distributed over the laminate. The person skilled in the art would realise that other bonding patterns are conceivable; for example the bonds can be arranged in groups or in bands. By this means, specific characteristics for the liquid-permeability can be achieved; for example bonding patterns that are band-shaped in the longitudinal direction of the laminate counteract liquid distribution perpendicular to these bands. The density of the bonds can also vary between different parts of the laminate, just as the laminate can comprise two or more different bonding patterns with different bond density and/or bonds with different forms. The bonds can also form patterns which are visually attractive to the user. Similarly, it is possible to divide the laminate into zones, where, for example, one zone has holes with an average cross-sectional area that is greater than the holes in another zone. This is particularly suitable when one part of the laminate is intended to receive low-viscous body fluids, while another part is primarily intended to receive faeces. The laminate can likewise comprise a zone without holes.

As is described in connection with FIG. 5b, it is possible to let only some of the recesses 5, 12 be provided with penetrating holes 9. This is particularly advantageous when it is desirable to obtain high liquid-permeability in only certain parts of the laminate 1, usually the central parts seen in the transverse direction of the laminate.

FIG. 3a shows a view from above of the bottom 6 of a recess 5 in the laminate 1, which bottom 6 constitutes an outer part of the intermediate wall 13 between the recesses 5, 12 in the first and second surfaces 8, 11. It can be seen here that a number of penetrating holes 9 are formed in the intermediate wall 13 in such a way that they form a net. Letting certain parts of the intermediate wall remain in this way not only achieves good liquid-permeability but also stabilises the recesses 5 and gives greater strength to the laminate, which reduces the risk of tearing during the manufacturing process and during use of the completed article. Another advantage of making several small holes 9 in each recess 5 is that the intermediate wall 13 will function as a sieve, which will retain, for example, faeces in the recesses while low-viscous body fluids will be allowed to pass through the holes 9. In a similar way, pulp, fibres and particles, for example superabsorbent particles, are retained inside the article by the intermediate wall 13. This effect has previously been achieved by means of a special intermediate layer, for example a tissue layer, arranged under the liquid-permeable surface layer. With the present invention, such intermediate layers will thus become superfluous, which gives a product that is simpler and cheaper to produce. As has been mentioned above, the invention is not limited to the cross-sections of the penetrating holes 9 shown here, the penetrating holes being able to have any cross-section in the plane of the laminate, such as circular, elongated or square with rounded corners. It is likewise understood that holes in one and the same recess can have different cross-sectional areas.

An alternative embodiment is shown in FIG. 3b, where only one penetrating hole 10 with a circular cross-section is formed in the intermediate wall 13. This embodiment is advantageous in that it gives good liquid-permeability. This hole 10, too, can have another cross-section than that shown, for example elongated, oval or square with rounded corners, and this hole, too, can be dimensioned to function as a sieve and particle barrier, as described above.

As an example that is in no way limiting, and with the intention of giving an understanding of the dimensions of the laminate, it can be mentioned that the surface layer suitably has a thickness before binding of 0.05-0.7 mm and the liquid transfer layer suitably has a thickness before binding of 0.5-15 mm. These values are obtained by means of standard test WSP120.6 (05). The method for measuring the thickness of a material web of nonwoven varies depending on the nature of layer. The method for measuring the thickness of a normal layer comprises the steps of applying a sample of the layer onto a reference plate and bringing a pressure plate under a pressure of 0.5 kPa into contact with the layer. The pressure plate has an area of ca 2500 mm² and the reference plate suitably has a diameter at least 50 mm greater than the diameter of the pressure plate. After 10 seconds the pressure is measured. The test is carried out on a total of 10 samples and the final thickness represents the mean value of these 10 tests. For bulky layers with a thickness less than 20 mm a device is used which comprises a vertical reference plate with an area of 1000 mm² and a vertical pressure plate with an area of 2500 mm², between which a sample is suspended, and a weighted lever, which is attached to the reference plate and applies a force to the reference plate in the direction towards the pressure plate with the purpose of separating two electrical contacts. The weight has a weight of 2.05±0.05 g, which gives a measuring pressure of 0.02 kPa. Prior to measuring, the pressure plate is led in the direction towards the sample until the electrical circuit is closed, which is indicated by a light bulb. After 10 seconds, the thickness of the sample is measured. The process is repeated nine times, after which a mean value of the thickness is calculated. Whether a layer is bulky or not is decided by means of a measuring device of the first type described above, wherein an average value of the thickness for 10 different samples is measured under pressures of 0.1 kPa and 0.5 kPa. If the sample material was compressed less than 20%, the layer is classed as normal, otherwise it is classed as bulky.

In an equally non-limiting way, it can be mentioned that the recesses suitably have a cross-sectional area at the intermediate wall of 0.0039-355 mm². A solitary penetrating hole formed in a recess advantageously has a cross-sectional area of 0.0039-355 mm², while the holes in a recess in which several penetrating holes are formed advantageously each have a cross-sectional area of 0.0039-40 mm². A particularly advantageous size for holes in a material web intended to receive body fluids is 1.57 mm². The cross-sectional areas of the holes and the recesses are measured by placing a sample of the material on a light table and then measuring the cross-sectional areas of the recesses and holes in the sample by means of a camera and a computer-generated image. It is suitable to measure 10-20 sizes and calculate a mean value for these.

In the case where the material web is divided into zones of the type described above, where one zone is intended to receive faeces and another zone to receive low-viscous body fluids, it is suitable if the holes in the former zone have a cross-sectional area of 9.5-355 mm² and the holes in the latter zone have a cross-sectional area of 0.0039-8 mm². It is of course suitable to adapt the recesses in a similar way, so that the recesses have in the former zone an average cross-section at the intermediate walls of 9.5-355 mm² and in the latter zone an average cross-section at the intermediate walls of 0.0039-8 mm². In the same way, it would be obvious to the person skilled in the art that an article intended primarily to receive body fluids is suitably provided with holes having a cross-sectional area of 0.0039-8 mm², while an article intended primarily to receive faeces is suitably provided with holes having a cross-sectional area of 9.5-355 mm².

It should also be pointed out that a material web in accordance with the invention is not limited to comprising a surface layer and a liquid transfer layer but can comprise one or several layers of different types, which in turn can comprise one or several strata displaying different characteristics.

FIG. 4 shows schematically a device 40 for producing a laminate 41 in accordance with the embodiments described above. The device 40 comprises an ultrasound welding arrangement, which in turn comprises a roll 46, to which a first and a second material web 42, 43 are fed, intended to constitute the surface layer and the liquid transfer layer of the laminate, respectively. The layers 42, 43 are each fed from a feeding roll 44, 45 in a transport direction indicated by the arrow A. The roll 46 is provided with a pattern of raised portions corresponding to the bonding and perforation patterns which are to be created by the process and are exemplified in FIGS. 3a and 3b. The ultrasound welding equipment further comprises a first and a second station 47, 48 located at the roll 46, which stations 47, 48 comprise a first and a second ultrasonic horn 49, 50, respectively, which are arranged in such a way along the transport direction of the layers 42, 43 that the layers 42, 43 first reach the first horn 49 and then the second horn 50. At the first horn 49 there occurs simultaneous compression and supply of energy to the layers 42, 43, whereupon the thermoplastic material existing in at least one of the layers 42, 43 in the laminate 41 is made to at least partly soften and bond the layers. In this way, a permanent compression or concentration of the porous structure is achieved, especially at the bonds, which creates recesses in the laminate 41, as has been described above in connection with FIGS. 1-3. When the two layers 42, 43 have been joined to each other in this way, the laminate 41 is fed forward to the second station 48 for forming penetrating holes in at least some of the recesses. At the perforation stage, the surface on the second ultrasonic horn 50 at the second station 48 that faces towards the laminate is advantageously knurled, in order to obtain good transfer of energy and more efficient perforation with minimal effect on the three-dimensional structure created in the first binding step. The laminate 41 is then guided forward, for example to be provided with further bonding patterns and bonds or to be incorporated into an absorbent article.

FIG. 5a shows how the ultrasonic horns in FIG. 4 are configured and arranged. Note that the second ultrasonic horn 50 has the same extension in the transverse direction of the laminate 41 as the first ultrasonic horn 49, so that penetrating holes are formed at all the bonds. FIG. 5b shows an alternative embodiment of the ultrasonic horns. The second ultrasonic horn 55 has a somewhat smaller extension in the transverse direction of the laminate 41 compared with the first ultrasonic horn 54, whereby a laminate is obtained with penetrating holes only in the recesses that are located within a part 53 that is centrally situated in the transverse direction of the laminate between the lines 57, 58. One of the advantages of an embodiment of this type is the prevention of liquid dispersion perpendicular to the longitudinal direction of the laminate. The embodiment in FIG. 5b is also advantageous when there is a desire to provide the side edges of the laminate with decorative patterns through the creation of bonds, as the bonds do not have to be provided with penetrating holes.

Although the devices in FIGS. 4, 5a and 5b only comprise two ultrasonic horns, it would be obvious to the person skilled in the art that further horns could be incorporated in the manufacturing process, either beside the existing horns seen in the transport direction of the laminate, or dispersed along the said transport direction. Thus, for example, parallel bands of bonds with holes can be achieved, separated by bands comprising recesses without such penetrating holes, and also bands completely lacking bonds can be produced. It is also possible to let the operational areas of the ultrasonic horns overlap each other, so that more than one ultrasonic horn at the first or the second station acts on a certain bond, in order to achieve a desired effect. It would also be obvious to the person skilled in the art that the ultrasonic horns shown in FIGS. 5a and b can have other extensions in the transverse direction of the laminate than those shown.

It is obvious to the person skilled in the art that other embodiments than those described above are conceivable. For example, the material web can comprise one, or more than two, material layers. It is also possible to form recesses in the material web without creating bonds between different layers. It is also possible to create recesses and holes in a material web using techniques other than ultrasound. For example, the holes and the recesses can be formed using hot calendering, in which case rolls with different extensions in the transverse direction of the material web can be used, in the same way as for the ultrasonic horns in FIG. 5b.

FIG. 6 shows an absorbent article 80 in the form of an incontinence protector, which comprises a laminate 81 in accordance with the invention, comprising a liquid-permeable surface layer 82, and a liquid-permeable liquid transfer layer 83. The liquid-permeable surface layer 82 contains, together with a liquid-impermeable surface layer 84, an absorbent body 85. The two surface layers 82, 84 have a somewhat greater extension in the plane than the absorbent body 85 and extend a distance beyond the edges of the absorbent body. The surface layers 82, 84 are mutually joined within the projecting parts 86, for example by gluing or welding with heat or ultrasound.

The absorbent body 85 can be of any conventional type. Examples of commonly occurring absorbent materials are cellulose fluff pulp, tissue sheets, highly absorbent polymers (so-called superabsorbents), absorbent foam materials, absorbent nonwoven materials and the like. It is common to combine cellulose fluff pulp with superabsorbents in an absorbent body. Absorbent bodies constructed of strata of different materials with different qualities with regard to liquid acquisition capacity, distribution capacity and storage capacity are also common. This is well-known to the person skilled in the art and therefore does not require to be described in detail. The thin absorbent bodies which are common nowadays in, for example, children's diapers and incontinence protectors often consist of a compressed, blended or layered structure of cellulose fluff pulp and superabsorbent.

On the outside of the liquid-impermeable surface layer 84 an attachment means 87 in the form of two longitudinal areas of self-adhesive glue is arranged. The areas of glue 87 are suitably covered before use with a detachable protective layer of paper or plastic film treated with releasing agent, which is not shown in the drawing. In the shown incontinence protector, this attachment means 87 consists of two longitudinal glue areas, however a number of other glue patterns are of course conceivable, as are other types of attachment means, such as hook-and-loop surfaces, press studs, girdles, special underpants, or the like.

An incontinence protector of the type shown in FIG. 6 is primarily intended to be used by persons with relatively slight incontinence problems and can easily be accommodated in a pair of ordinary underpants. The attachment means 87 serves to hold the incontinence protector in place in the underpants during use.

The incontinence protector 80 is hour-glass shaped with broader end portions 88, 89 and a narrower crotch portion 90 situated between the end portions 88, 89. The crotch portion 90 is the part of the incontinence protector 80 that is intended during use to be placed in the user's crotch and to serve as an acquisition surface for the emitted body fluid.

A porous and resilient liquid transfer layer 83, for example a fibrous wadding, a porous foam layer, or one of the other materials that have been indicated as suitable for the second layer in the laminate described above, is arranged between the liquid-permeable surface layer 82 and the absorbent body 85. The liquid transfer layer 83 receives the liquid that passes through the surface layer 82. In the case of urination, relatively large quantities of liquid are often emitted during short periods. Therefore, it is essential that the contact between the liquid-permeable surface area 82 and the liquid transfer layer 83 behind it is such that the liquid penetrates quickly into the liquid transfer layer 83. Due to the fact that the liquid transfer layer 83 is a layer with high bulk and a thickness that is preferably from 0.5 mm-3 mm, the layer can act as a temporary reservoir for liquid before it is gradually absorbed into the absorbent body 85.

In the shown example, the liquid transfer layer 83 is somewhat narrower than the absorbent body 85, but extends along the entire length of the incontinence protector 80. This type of design is advantageous as it allows a certain saving in material. Naturally, it is possible to make further savings in material by not letting the liquid transfer layer extend along the entire length of the incontinence protector. For example, it is conceivable to only arrange the liquid transfer layer at the crotch portion 90 of the incontinence protector, as the bulk of the body fluid to be absorbed by the incontinence protector can be expected to meet the protector within that portion.

Commonly used liquid transfer layers are often very porous and have a relatively large effective average pore size, which is often greater than the effective average pore size of conventional liquid-permeable surface layer materials. The effective average pore size of a fibrous material can be measured using a measuring method described in EP-A-0 470 392. As liquid, due to capillary action, strives to go from courser to finer capillaries, and not vice versa, liquid tends to remain in the fibre network of the surface material instead of being drained by the more porous liquid transfer layer. This means that there is a risk of liquid running on the surface of the surface layer and causing leakage. Moreover, liquid remains in the fibre structure of the surface layer, causing the surface of the surface layer to feel wet and uncomfortable to the user.

As described above, the liquid-permeable surface layer 82 and the liquid transfer layer 83 constitute a laminate 81, which comprises recesses with penetrating holes through the laminate. These holes thus lead the body fluid from the surface layer 82 and a first surface of the laminate 81 in a direction towards the absorbent body 85 and a second surface of the laminate 81. Furthermore, as a result of the liquid-permeable surface layer 82 being joined to the liquid transfer layer 83, as described in connection with the laminates described above, the liquid transfer layer is compressed at the bonds 91. Thus, the liquid transfer layer 83 has a density gradient with increasing density in towards each bond 91. The liquid transfer layer 83 will thus have a pore size gradient around the bonds 91 and an area where the effective average pore size is less than the average pore size of the liquid-permeable surface layer 82. Thus, the liquid transfer layer 83 can efficiently drain the surface layer 82 of liquid. As the surface layer 82 is drained of liquid in the area around each bond 91, a liquid deficit will occur in these areas, whereupon a levelling-out of liquid will occur in the surrounding areas. The surface layer 82 will then contain less liquid altogether and will thus feel drier against the skin.

Moreover, the absorbent body 85 should have greater liquid affinity than the liquid transfer layer 83, in order to achieve good liquid transfer between the liquid transfer layer 83 and the absorbent body 85. This can be achieved, for example, by means of the absorbent body 85 having a finer capillary structure than the liquid transfer layer 83 and/or by the liquid transfer layer 83 being less hydrophilic than the absorbent body 85. In the same way, it is advantageous if the liquid transfer layer is more hydrophilic than the surface layer, thus obtaining a hydrophilic gradient, which gives greater hydrophilicity in a direction from the surface layer towards the absorbent body. Rewetting of the surface layer is thus prevented and good liquid transfer into the absorbent body is ensured. Thus, as the surface layer is advantageously relatively hydrophobic, it is particularly advantageous that the recesses be formed with penetrating holes in order to ensure good liquid transfer from the surface layer to the liquid transfer layer. Hydrophilicity in hydrophobic materials, such as the thermoplastic materials in the laminate, is suitably achieved by means of treatment with surfactants, in a manner known to the person skilled in the art.

As has been mentioned above, it is suitable if the recesses with holes are formed with a number of small holes, as this gives a filter effect which prevents fibres and particles from leaving the article. A separator in the form of an intermediate layer thus becomes unnecessary in such an embodiment.

Obviously, it is possible to use a material web in accordance with the present invention in many different types of absorbent article, such as diapers, sanitary napkins, incontinence protectors, protective bed covers, etc.

Moreover, as mentioned above, a material web in accordance with the invention is not limited to comprising a surface layer and a liquid transfer layer; the material web can comprise several layers of different types. Equally, the material web can comprise only one layer and the layers can, in turn have several different characteristics in different layers. Neither does the material web have to be arranged as shown in FIG. 6, but it can be located anywhere in the absorbent article. The material web can also be arranged in such a way that the first surface is facing away from the user during use of the article, just as it can be facing towards the user.

As mentioned above, the material web can also be divided into zones with holes and recesses of different sizes. The material web can then, for example, be so arranged in the article that a zone intended to receive faeces is closer to a rear edge of the article, while a zone intended to receive low-viscous body fluids is closer to a forward edge of the article.

The invention should not be considered to be limited to the embodiments described here, a number of further variations and modifications being conceivable within the framework of the following claims, and it is also possible to combine features from different embodiments. One example of such a combination comprises an incontinence protector comprising a material web consisting of only one layer, which layer is divided into a zone with penetrating holes and a zone without penetrating holes. Another example is constituted by a material web comprising three layers, which material web has three zones in which the holes have different cross-sectional areas.

1. Material web for use in an absorbent article, the material web comprises at least one fibrous material layer and has a longitudinal direction, a transverse direction and a thickness direction as well as a first and a second surface, said surfaces are situated on opposite sides of the material web and one of said surfaces is intended to face towards a user of the article while the opposite surface is intended to face away from a user of the article, the material web further comprising:
recesses in the first surface with an extension in the thickness direction of the material web, which recesses have a diminishing cross-sectional area along at least a part of their extension in a direction towards the second surface;
recesses in the second surface with an extension in the thickness direction of the material web, which recesses have a diminishing cross-sectional area along at least a part of their extension in a direction towards the first surface and which recesses form pairs with opposite recesses in the first surface;
the recesses in at least some of the pairs of recesses being connected to each other via at least one hole;
wherein the material web comprises at least a first and a second fibrous material layer, which are bonded together at the recesses by means of an at least partly softened thermoplastic material, wherein the first fibrous material layer is a surface layer having a thickness before binding of 0.05-0.7 mm measured according to WSP120.6 (05) and the second fibrous material layer is a liquid transfer layer having a thickness before binding of 0.5-15 mm measured according to WSP120.6 (05).

2. Material web in accordance with claim 1, wherein the recesses in at least some of the pairs of recesses are connected to each other via several holes.

3. Material web in accordance with claim 1, further comprising a first and a second zone, wherein holes within the first zone have an average cross-sectional area that is smaller than the average cross-sectional area for holes within the second zone.

4. Material web in accordance with claim 1, wherein the recesses in most or all of the pairs of recesses are connected via at least one hole.

5. Material web in accordance with claim 1, wherein the recesses provided with holes are situated within a part that is situated centrally in the transverse direction of the material web.

6. Absorbent article comprising a liquid-permeable surface layer, a liquid-impermeable surface layer and an absorbent layer between the two surface layers, wherein said liquid-permeable surface layer and a liquid-permeable liquid transfer layer arranged between the liquid-permeable surface layer and the absorbent layer are in the form of a material web in accordance with claim 1.

7. Absorbent article in accordance with claim 6, wherein the article, in the longitudinal direction, has a forward transverse edge and a rear transverse edge and wherein the material web has a first zone situated closer to the forward edge and a second zone situated closer to the rear edge, wherein holes situated within the first zone have an average cross-sectional area that is smaller than the average cross-sectional area for holes within the second zone.

8. Method for producing an apertured structure in a material web for use in an absorbent article, the material web comprises at least one fibrous material layer and has a longitudinal direction, a transverse direction and a thickness direction as well as a first and a second surface, the surfaces are situated on opposite sides of the material web and one of said surfaces is intended to face towards a user of the article, while the opposite surface is intended to face away from a user of the article, the method comprising:
a first step of forming pairs of mutually opposite recesses in the first and second surfaces, the recesses have an extension in the thickness direction of the material web, the recesses in the first surface being formed with a diminishing cross-sectional area at least along part of their extension in a direction towards the second surface and the recesses in the second surface being formed with a diminishing cross-sectional area at least along part of their extension in a direction towards the first surface; and
a second step of forming holes in the material web, the holes each connect two recesses belonging to a pair, wherein said recesses and holes are formed by means of an ultrasonic welding device comprising at least one first ultrasonic horn for obtaining said recesses, at least one second ultrasonic horn for obtaining said holes, and a roll, wherein said first and second ultrasonic horns work against said roll.

9. Method in accordance with claim 8, wherein the recesses in at least some of the pairs of recesses are connected by means of several holes.

10. Method in accordance with claim 8, wherein the material web comprises at least a first and a second material layer, of which at least one layer comprises a thermoplastic material, and wherein during the forming of the recesses said thermoplastic material is made to at least partly soften and thus bond together the two material layers at the recesses.

11. Method in accordance with claim 8, wherein holes within a first zone of the material web are formed with an average cross-sectional area that is smaller than the average cross-sectional layer for holes within a second zone of the material web.

12. Method in accordance with claim 8, wherein the holes connect recesses situated within a part that is situated centrally in the transverse direction of the material web.
